(19) United States
(12) Patent Application Publication
(10) Pub. No.: US 2017/0311843 A1
(43) Pub. Date: Nov. 2, 2017

(54) MEDICAL INSTRUMENT TRACKING INDICATOR SYSTEM

(71) Applicants: Brent Andrew BAILEY, Toronto (CA); Kirusha SRIMOHANARAJAH, Toronto (CA)

(72) Inventors: Brent Andrew BAILEY, Toronto (CA); Kirusha SRIMOHANARAJAH, Toronto (CA)

(21) Appl. No.: 15/139,620

(22) Filed: Apr. 27, 2016

(51) Int. Cl. A61B 5/06; A61B 90/00; A61B 5/00

(52) U.S. Cl. CPC A61B 5/064; A61B 5/742; A61B 90/361; A61B 90/39; A61B 2090/3937

(57) ABSTRACT

In one aspect there is provided a medical navigation system, comprising a computing device having a processor coupled to a memory, a tracking camera for tracking medical instruments, and a display for displaying an image. Tracked medical instruments may have attached thereto an indicator device having a visual indicator. The indicator device is positioned in view of a user during the medical procedure, and is configured to receive, from the medical navigation system, data indicating a quality of the tracking of the one or more instruments, and to display, using the visual indicator, the data indicating the quality of the tracking.

### TECHNICAL FIELD

The present disclosure generally relates a medical instrument tracking indicator system for use during medical procedures.

### BACKGROUND

In the field of medicine, imaging and image guidance are a significant component of clinical care. From diagnosis and monitoring of disease, to planning of the surgical approach, to guidance during procedures and follow-up after the procedure is complete, imaging and image guidance provides effective and multifaceted treatment approaches, for a variety of procedures, including surgery and radiation therapy. Targeted stem cell delivery, adaptive chemotherapy regimens, and radiation therapy are only a few examples of procedures utilizing imaging guidance in the medical field.

Advanced imaging modalities such as Magnetic Resonance Imaging (“MRI”) have led to improved rates and accuracy of detection, diagnosis and staging in several fields of medicine including neurology, where imaging of diseases such as brain cancer, stroke, Intra-Cerebral Hemorrhage (“ICH”), and neurodegenerative diseases, such as Parkinson's and Alzheimer's, are performed. As an imaging modality, MRI enables three-dimensional visualization of tissue with high contrast in soft tissue without the use of ionizing radiation. This modality is often used in conjunction with other modalities such as Ultrasound (“US”), Positron Emission Tomography (“PET”) and Computed X-ray Tomography (“CT”), by examining the same tissue using the different physical principals available with each modality. CT is often used to visualize boney structures, and blood vessels when used in conjunction with an intra-venous agent such as an iodinated contrast agent. MRI may also be performed using a similar contrast agent, such as an intra-venous gadolinium based contrast agent which has pharmaco-kinetic properties that enable visualization of tumors, and break-down of the blood brain barrier. These multi-modality solutions can provide varying degrees of contrast between different tissue types, tissue function, and disease states. Imaging modalities can be used in isolation, or in combination to better differentiate and diagnose disease.

In neurosurgery, for example, brain tumors are typically excised through an open craniotomy approach guided by imaging. The data collected in these solutions typically consists of CT scans with an associated contrast agent, such as iodinated contrast agent, as well as MRI scans with an associated contrast agent, such as gadolinium contrast agent. Also, optical imaging is often used in the form of a microscope to differentiate the boundaries of the tumor from healthy tissue, known as the peripheral zone.

Tracking of medical instruments relative to the patient and the associated imaging data is also often achieved by way of external hardware systems such as mechanical arms, or radiofrequency or optical tracking devices. As a set, these devices are commonly referred to as surgical navigation systems.

### SUMMARY

During a medical procedure, the surgical navigation system may operate to continuously track medical instruments relative to the patient and the associated imaging data. However, the tracking of a medical instrument may partially or completely fail momentarily or for an extended period of time for any number of reasons. For example, the surgical navigation system may lose communication with one or more tracked instruments. In one example, the surgical navigation system utilizing an optical tracking using a camera may lose communication with one or more tracked instruments if the light-of-sight between the tracked instrument and the optical tracking device is lost. This may occur during a medical procedure as a user (e.g. a surgeon) operating the tracked instrument moves the instrument while focusing on the patient. Accordingly, there is a need for an improved medical instrument tracking indicator system to alert the user of the quality of the tracking.

One aspect of the present disclosure, a medical navigation system for use during a medical procedure, the medical navigation system comprising a tracking camera configured to track one or more instruments during the medical procedure, the one or more instruments being in communication with the tracking camera; and an indicator device attached to at least one of the one or more instruments, and having a visual indicator positioned in view of a user during the medical procedure, the indicator device being configured to receive, from the medical navigation system, data indicating a quality of the tracking of the one or more instruments, and to display, using the visual indicator, the data indicating the quality of the tracking.

In another aspect of the present disclosure, a method for providing indicator data during a medical procedure, the method comprising tracking, using a tracking camera, one or more instruments during the medical procedure, at least one of the one or more instruments having an indicator device attached thereto and having a visual indicator positioned in view of a user during the medical procedure; determining a quality of the tracking of the one or more instruments; sending, to the indicator device, indicator data indicating the quality of the tracking of one or more instruments; and displaying, on the visual indicator, the quality of the tracking of the one or more instruments.

A further understanding of the functional and advantageous aspects of the disclosure can be realized by reference to the following detailed description and drawings.

### BRIEF DESCRIPTION OF THE DRAWINGS

Embodiments will now be described, by way of example only, with reference to the drawings, in which:

FIG. 1 illustrates the insertion of an access port into a human brain, for providing access to internal brain tissue during a medical procedure;

FIG. 2 shows an exemplary navigation system to support minimally invasive access port-based surgery;

FIG. 3A is a block diagram illustrating a control and processing system that may be used in the navigation system shown in FIG. 2;

FIG. 3B is a block diagram illustrating an indicator device that may be used in the navigation system shown in FIG. 2;

FIG. 3C shows an exemplary indicator device of FIG. 3B in operation;

FIG. 4A is an exemplary navigation system similar to FIG. 2 illustrating system components of an exemplary surgical system used in port based surgery;

FIG. 4B is an exemplary embodiment illustrating various detailed aspects of a port based surgery as seen in FIG. 1;

FIG. 4C is an exemplary access port utilizing the indicator device of FIG. 3B;

FIG. 4D is an exemplary clinical tool utilizing the indicator device of FIG. 3B;

FIG. 4E is an exemplary tracking camera for use with exemplary navigation system of FIG. 2 utilizing the indicator device of FIG. 3B;

FIG. 5A is an exemplary navigation system similar to FIG. 2 illustrating system components of an exemplary surgical system used in port based surgery;

FIG. SB is an exemplary end effector utilizing the indicator device of FIG. 3B for use with exemplary navigation system of FIG. 5A; and

FIG. 6 is an exemplary flowchart of a method for providing indicator data to the indicator device of FIG. 3B.

Similar reference numerals are used in different figures to denote similar components.

### DETAILED DESCRIPTION

Various embodiments and aspects of the disclosure will be described with reference to details discussed below. The following description and drawings are illustrative of the disclosure and are not to be construed as limiting the disclosure. Numerous specific details are described to provide a thorough understanding of various embodiments of the present disclosure. However, in certain instances, well-known or conventional details are not described in order to provide a concise discussion of embodiments of the present disclosure.

As used herein, the terms, “comprises” and “comprising” are to be construed as being inclusive and open ended, and not exclusive. Specifically, when used in the specification and claims, the terms, “comprises” and “comprising” and variations thereof mean the specified features, steps or components are included. These terms are not to be interpreted to exclude the presence of other features, steps or components.

As used herein, the term “exemplary” means “serving as an example, instance, or illustration,” and should not be construed as preferred or advantageous over other configurations disclosed herein.

As used herein, the terms “about”, “approximately”, and “substantially” are meant to cover variations that may exist in the upper and lower limits of the ranges of values, such as variations in properties, parameters, and dimensions. In one non-limiting example, the terms “about”, “approximately”, and “substantially” mean plus or minus 10 percent or less.

Unless defined otherwise, all technical and scientific terms used herein are intended to have the same meaning as commonly understood by one of ordinary skill in the art. Unless otherwise indicated, such as through context, as used herein, the following terms are intended to have the following meanings:

As used herein, the phrase “access port” refers to a cannula, conduit, sheath, port, tube, or other structure that is insertable into a subject, in order to provide access to internal tissue, organs, or other biological substances. In some embodiments, an access port may directly expose internal tissue, for example, via an opening or aperture at a distal end thereof, and/or via an opening or aperture at an intermediate location along a length thereof. In other embodiments, an access port may provide indirect access, via one or more surfaces that are transparent, or partially transparent, to one or more forms of energy or radiation, such as, but not limited to, electromagnetic waves and acoustic waves.

As used herein the phrase “intraoperative” refers to an action, process, method, event or step that occurs or is carried out during at least a portion of a medical procedure. Intraoperative, as defined herein, is not limited to surgical procedures, and may refer to other types of medical procedures, such as diagnostic and therapeutic procedures.

Embodiments of the present disclosure provide imaging devices that are insertable into a subject or patient for imaging internal tissues, and methods of use thereof.

Some embodiments of the present disclosure relate to minimally invasive medical procedures that are performed via an access port, whereby surgery, diagnostic imaging, therapy, or other medical procedures (e.g. minimally invasive medical procedures) are performed based on access to internal tissue through the access port.

The present disclosure is generally related to medical procedures, neurosurgery, and minimally invasive port-based surgery in specific.

In the example of a port-based surgery, a surgeon or robotic surgical system may perform a surgical procedure involving tumor resection in which the residual tumor remaining after is minimized, while also minimizing the trauma to the healthy white and grey matter of the brain. In such procedures, trauma may occur, for example, due to contact with the access port, stress to the brain matter, unintentional impact with surgical devices, and/or accidental resection of healthy tissue. A key to minimizing trauma is ensuring that the spatial location of the patient as understood by the surgeon and the surgical system is as accurate as possible.

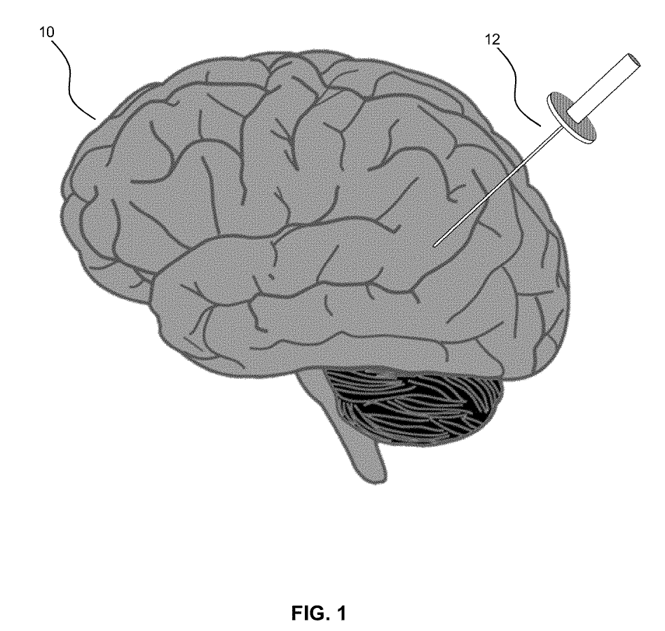

FIG. 1 illustrates the insertion of an access port into a human brain, for providing access to internal brain tissue during a medical procedure. In FIG. 1, access port 12 is inserted into a human brain 10, providing access to internal brain tissue. Access port 12 may include instruments such as catheters, surgical probes, or cylindrical ports such as the NICO BrainPath™. Surgical tools and instruments may then be inserted within the lumen of access port 12 in order to perform surgical, diagnostic or therapeutic procedures, such as resecting tumors as necessary. The present disclosure applies equally well to catheters, deep brain simulation (‘DBS’) needles, a biopsy procedure, and also to biopsies and/or catheters in other medical procedures performed on other parts of the body where head immobilization is needed.

In the example of a port-based surgery, a straight or linear access port 12 is typically guided down a sulci path of the brain. Surgical instruments would then be inserted down the access port 12.

Optical tracking systems, which may be used in the medical procedure, track the position of a part of the instrument that is within line-of-site of the optical tracking camera. The optical tracking camera has a field of view limited by the optics, sensors, and programming of the camera. Only objects in the field of view of the optical tracking camera are within the line-of-sight of the optical tracking camera.

Optical tracking systems also require a reference to the patient to know where the instrument is relative to the target (e.g., a tumor) of the medical procedure. These optical tracking systems require a knowledge of the dimensions of the instrument being tracked so that, for example, the optical tracking system knows the position in space of a tip of a medical instrument relative to the tracking markers being tracked.

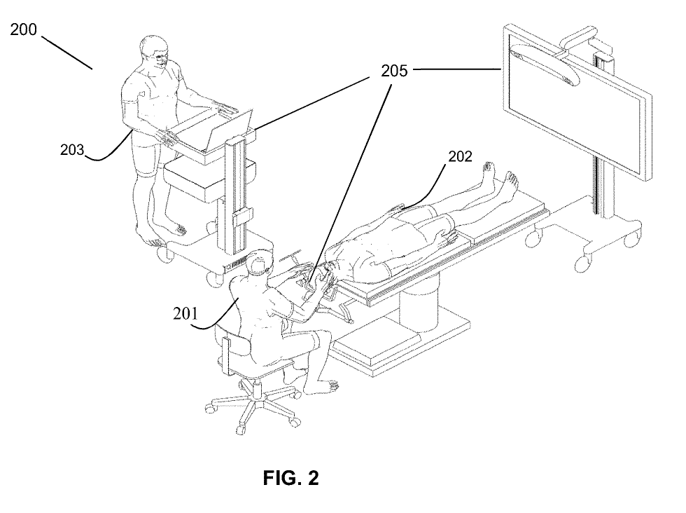

Referring to FIG. 2, an exemplary navigation system environment 200 is shown, which may be used to support navigated image-guided surgery. As shown in FIG. 2, user (surgeon) 201 conducts a surgery on a patient 202 in an operating room (OR) environment. A medical navigation system 205 comprising an equipment tower, tracking system, displays and tracked instruments assist the surgeon 201 during his procedure. An operator 203 is also present to operate, control and provide assistance for the medical navigation system 205.

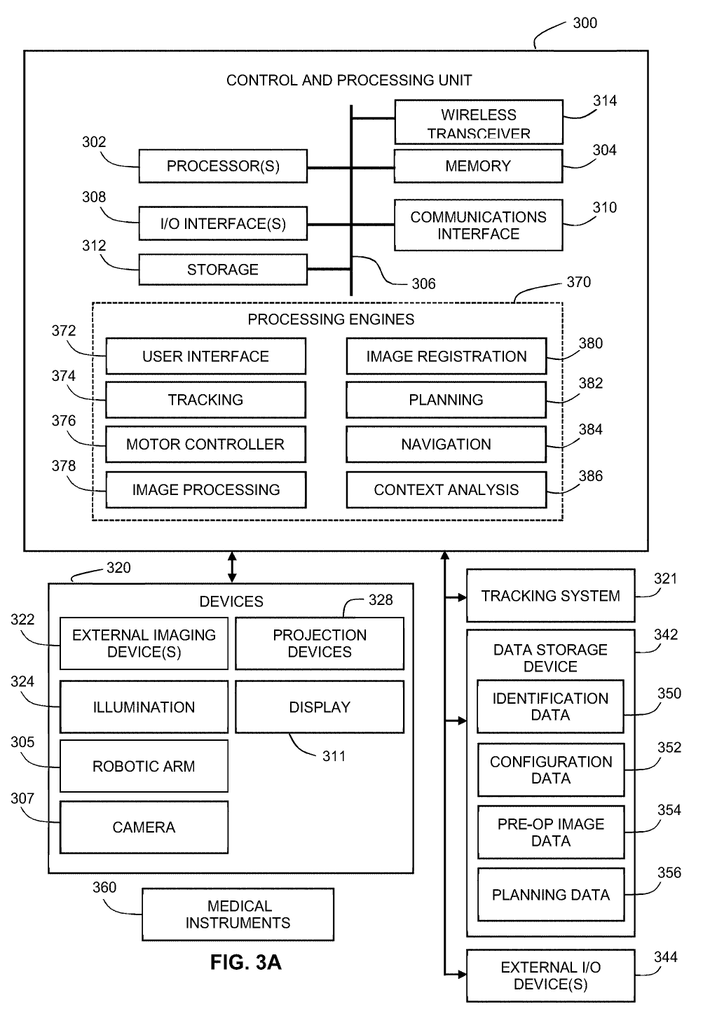

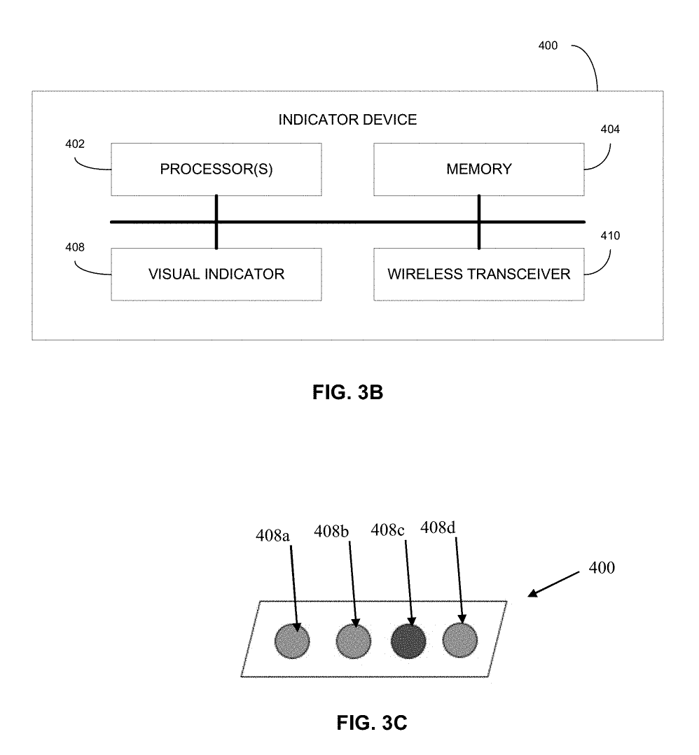

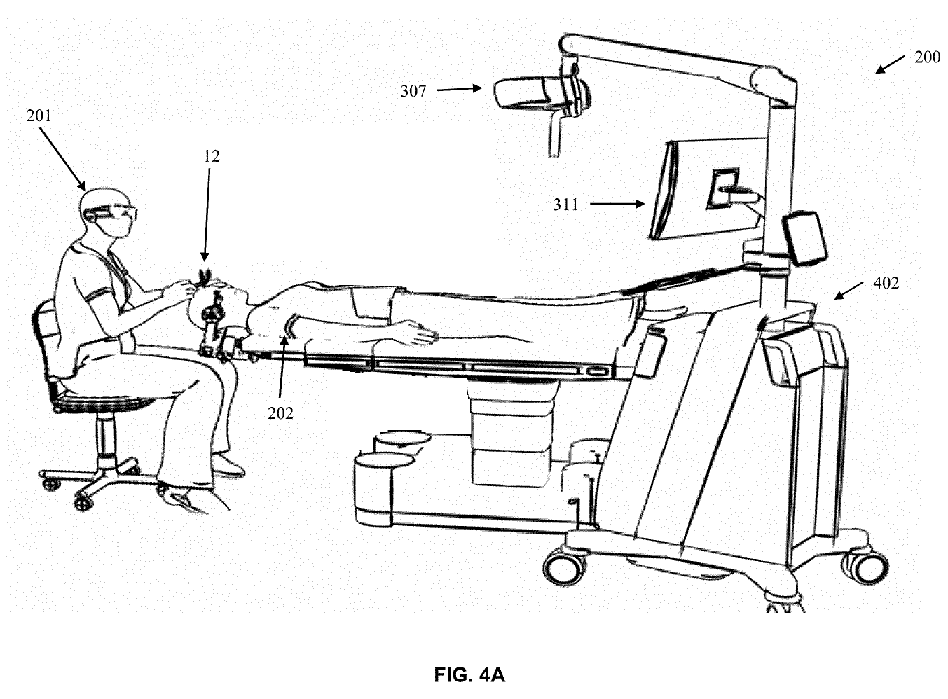

Referring to FIG. 3A, a block diagram is shown illustrating a control and processing system 300 that may be used in the medical navigation system 205 shown in FIG. 2 (e.g., as part of the equipment tower). As shown in FIG. 3A, in one example, control and processing system 300 may include one or more processors 302, a memory 304, a system bus 306, one or more input/output interfaces 308, a communications interface 310, storage device 312, and wireless transceiver 314. Control and processing system 300 may be interfaced with other external devices, such as tracking system 321, data storage 342, and external user input and output devices 344, which may include, for example, one or more of a display, keyboard, mouse, sensors attached to medical equipment, foot pedal, and microphone and speaker.

Data storage 342 may be any suitable data storage device, such as a local or remote computing device (e.g. a computer, hard drive, digital media device, or server) having a database stored thereon. In the example shown in FIG. 3A, data storage device 342 includes identification data 350 for identifying one or more medical instruments 360 and configuration data 352 that associates customized configuration parameters with one or more medical instruments 360. Data storage device 342 may also include preoperative image data 354 and/or medical procedure planning data 356. Although data storage device 342 is shown as a single device in FIG. 3A, it will be understood that in other embodiments, data storage device 342 may be provided as multiple storage devices.

Medical instruments 360 are identifiable by control and processing unit 300. Medical instruments 360 may be connected to and controlled by control and processing unit 300, or medical instruments 360 may be operated or otherwise employed independent of control and processing unit 300. Tracking system 321 may be employed to track one or more of medical instruments 360 and spatially register the one or more tracked medical instruments to an intraoperative reference frame. For example, medical instruments 360 may include tracking markers such as tracking spheres that may be recognizable by a tracking camera 307. In one example, the tracking camera 307 may be an infrared (IR) tracking camera. In another example, as sheath placed over a medical instrument 360 may be connected to and controlled by control and processing unit 300.

Control and processing unit 300 may also interface with a number of configurable devices, and may intraoperatively reconfigure one or more of such devices based on configuration parameters obtained from configuration data 352. Examples of devices 320, as shown in FIG. 3A, include one or more external imaging devices 322, one or more illumination devices 324, a robotic arm 305, one or more projection devices 328, and one or more displays 311.

Exemplary aspects of the disclosure can be implemented via processor(s) 302 and/or memory 304. For example, the functionalities described herein can be partially implemented via hardware logic in processor 302 and partially using the instructions stored in memory 304, as one or more processing modules or engines 370. Example processing modules include, but are not limited to, user interface engine 372, tracking module 374, motor controller 376, image processing engine 378, image registration engine 380, procedure planning engine 382, navigation engine 384, and context analysis module 386. While the example processing modules are shown separately in FIG. 3A, in one example the processing modules 370 may be stored in the memory 304 and the processing modules may be collectively referred to as processing modules 370.

It is to be understood that the system is not intended to be limited to the components shown in FIG. 3A. One or more components of the control and processing system 300 may be provided as an external component or device. In one example, navigation module 384 may be provided as an external navigation system that is integrated with control and processing system 300.

Some embodiments may be implemented using processor 302 without additional instructions stored in memory 304. Some embodiments may be implemented using the instructions stored in memory 304 for execution by one or more general purpose microprocessors. Thus, the disclosure is not limited to a specific configuration of hardware and/or software.

While some embodiments can be implemented in fully functioning computers and computer systems, various embodiments are capable of being distributed as a computing product in a variety of forms and are capable of being applied regardless of the particular type of machine or computer readable media used to actually effect the distribution.

According to one aspect of the present application, one purpose of the navigation system 205, which may include control and processing unit 300, is to provide tools to the neurosurgeon that will lead to the most informed, least damaging neurosurgical operations. In addition to removal of brain tumors and intracranial hemorrhages, the navigation system 205 can also be applied to a brain biopsy, a functional/deep-brain stimulation, a catheter/shunt placement procedure, open craniotomies, endonasal/skull-based/ENT, spine procedures, and other parts of the body such as breast biopsies, liver biopsies, etc. While several examples have been provided, aspects of the present disclosure may be applied to any suitable medical procedure.

While one example of a navigation system 205 is provided that may be used with aspects of the present application, any suitable navigation system may be used, such as a navigation system using optical tracking instead of infrared cameras.

Referring to FIG. 3B, a block diagram is shown illustrating an indicator device 400, that may be used in the medical navigation system 205 shown in FIG. 2. As shown in FIG. 3B, in one example, indicator device 400 may include processor(s) 402, memory 404, a visual indicator 408, and a wireless transceiver 410. Indicator device 400 may also include a battery to provide power to the various components of the device (not shown). Visual indicator 408 may be implemented using any number of visual devices, for example, one or more light-emitting diodes ('LEDs'), an LED display device (e.g. LED display, liquid crystal display (LCD), organic light-emitting diode display (OLED), segment display, or other type of display). Wireless transceiver 410 may be configured to communicate with wireless transceiver 314 of control and processing system 300 of medical navigation system 205. Accordingly, indicator device 400 is configured to send and receive data to and from medical navigation system 205. The wireless transceivers 410, 314 may communicate via known wireless communication protocols (e.g. Wi-Fi or Bluetooth™). Data received at wireless transceivers 410 may be processed by processor 402 in accordance with program instructions stored in memory 404, and the processed data may be displayed using visual indicator 408.

A single indicator device 400 may be attached to one medical instrument 360 in use in the medical navigation system 205. In some instances an indicator device 400 is attached to each medical instrument 360 in use in the medical navigation system 205.

Indicator device 400 is positioned such that the device is in view of user 201 (e.g. surgeon) using the medical instrument 360 during a medical procedure, and whilst the medical instrument 360 is in communication with the tracking camera 307.

Shown in FIG. 3C is an example embodiment of indicator device 400 having four visual indicators 408a, 408b, 408c, and 408c. As shown, visual indicators 408a-408d are multi-colored LEDs. Indicator device 400 receives from medical navigation system 205 data indicating data indicating quality of the tracking of one or more tracked instruments to indicator devices 400. Indicator device 400 controls the color of each visual indicator based on the received data. For example, a red color indicates poor tracking quality and a green color represents good tracking quality. Since indicator device 400 has four visual indicators (408a-408d), indicator device 400 can display data indicating quality of the tracking for up to four tracked instruments simultaneously. In other embodiments, more or fewer visual indicators can be used.

In one embodiment, visual indicator 408a indicates the quality of tracking of a patient reference instrument, visual indicator 408b indicates the quality of tracking of a pointer tool, visual indicator 408c indicates the quality of tracking of a reference array, and visual indicator 408d indicates the quality of tracking of a suction instrument. As shown in FIG. 3C, the patient reference instrument, the pointer tool, and the suction instrument have good quality tracking, whereas the reference array has poor quality tracking. The information provided therefore consolidates information from multiple devices into a single location that is in view of user 201 (surgeon) during the medical procedure. Accordingly, user 201 does not have to interrupt the medical procedure to inquire about the status of the tracking. Further, if tracking quality is an important feature for the medical procedure, user 201 may adjust instruments held by him to ensure that the instruments are properly tracked. Alternatively, user 201 may choose to focus on the medical procedure and reduce their reliance on the tracking of the instrument that has poor tracking quality.

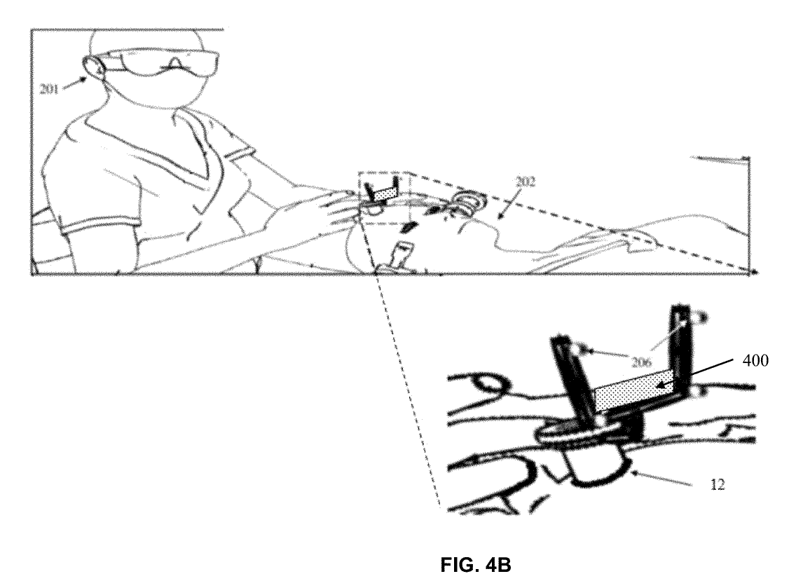

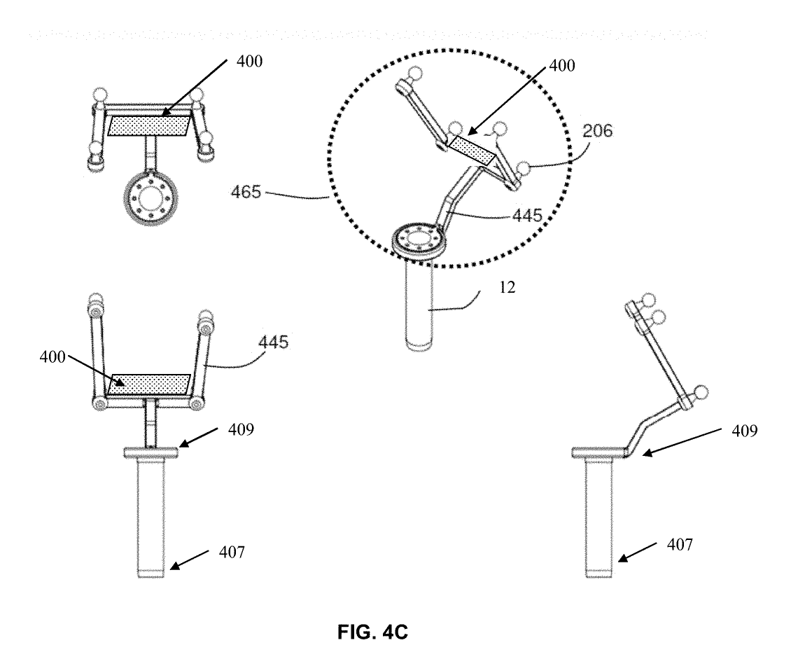

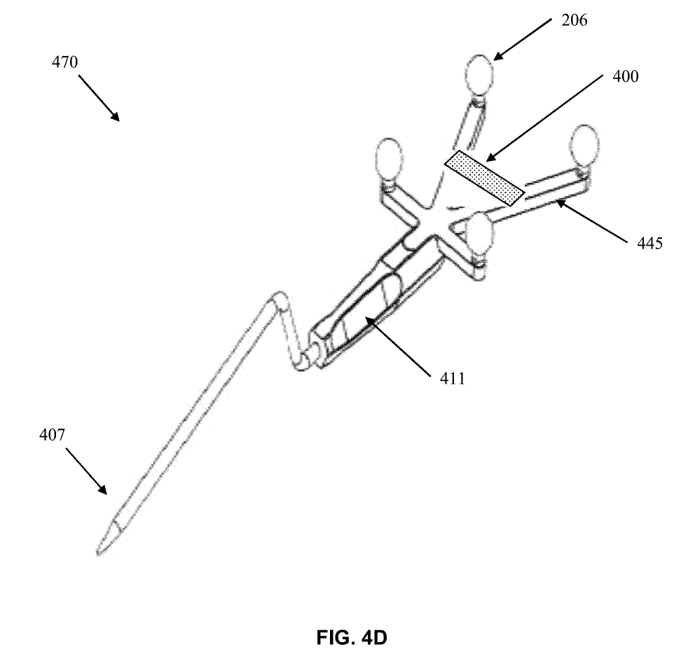

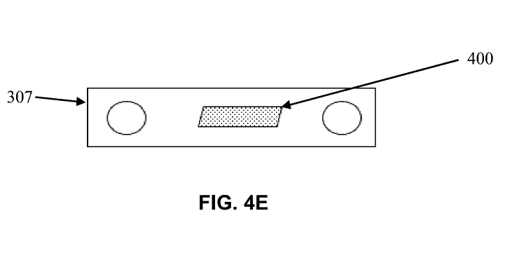

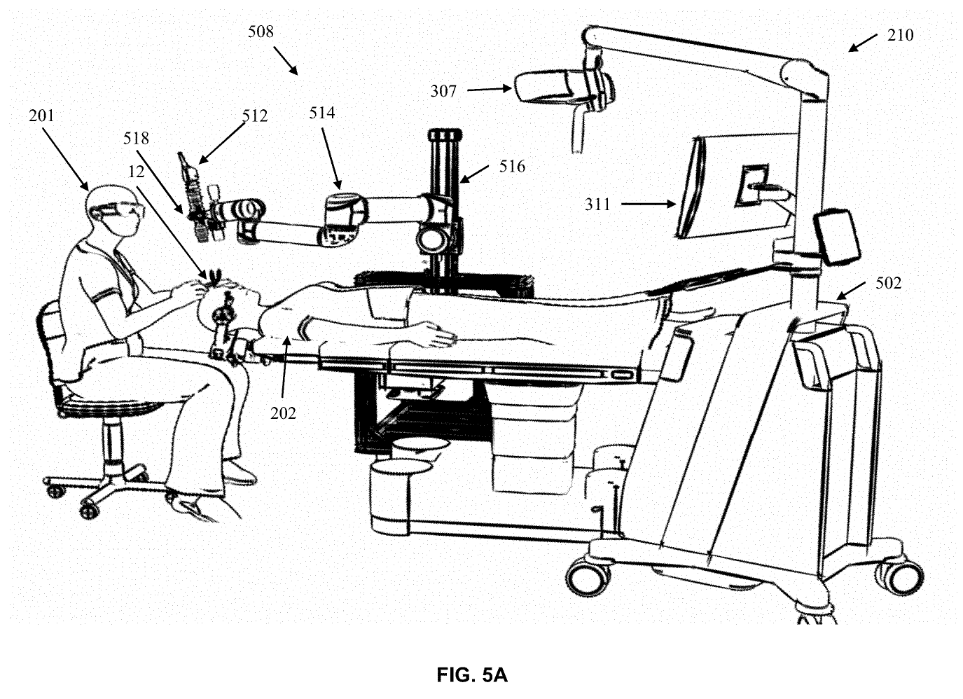

FIG. 4A and FIG. 4B show exemplary navigation system environment 200 in which indicator device 400 may operate. As shown, navigation system environment 200 includes components of medical navigation system 205, including equipment tower 402, tracking camera 307, and display 311. As shown, patient 202 is prepped for a medical procedure to be carried on by user 201 (e.g. a surgeon). User 201 may utilize various medical instruments 360 during the medical procedure, such as access port 12 (as shown in FIG. 4C) or other clinical tool 470 (as shown in FIG. 4D). Medical instruments 360 may have attached thereto an indicator device 400 to provide indicator data to user 201 during medical procedures. Each medical instrument 360 (such as access port 12 and other clinical tool(s) 470) operating in navigation system environment 200 may be tracked by tracking camera 307 and tracking system 321. Furthermore, indicator device 400 may display, on a visual indicator 408, the quality of the tracking of one or more medical instruments 360.

As illustrated in FIGS. 4A-4D, tracking markers 206 may be fitted to medical instruments 360, including access port 12 and clinical tool 470, to aid tracking by tracking camera 307 and tracking system 321. Medical navigation system 205 determines the spatial position and pose of medical instruments 360 using tracking camera 307, which senses tracking markers 206 that are in the field of view of the camera. Medical navigation system 205 thus registers the position of tracked medical instruments 360 within a common coordinate frame.

In one embodiment, passive tracking markers such as the reflective spherical markers 206 shown in FIGS. 4B-4D are seen by the tracking camera 307 to give identifiable points for spatially locating and determining the pose of a tracked instrument to which tracking markers 260 are attached (for example a port 12 or clinical tool 470). To successfully track a medical instrument, at least three of the tracking markers 260 attached to the medical instrument must be positioned at least partially within the field of view of tracking camera 307. A medical instrument that is successfully tracked is in communication with tracking camera 307. Tracking camera 307 and medical navigation system 205 may be able to identify the spatial position, pose, and 3D volume representation of the instrument 360 within the common coordinate frame.

As seen in FIG. 4C and FIG. 4D, each medical instrument 360 (such as port 12 and clinical tool 470) may have a unique, marker assembly 465 attached rigidly thereto. The uniqueness of the marker assembly 460 may be used to identify the corresponding medical instrument. For example, each instrument may have a specific orientation of tracking markers 260 relative to one another on a marker assembly 445. Accordingly, each tracked instrument will have a unique individual identity within navigation system 200. Navigation system 205 may include a database of medical instruments including data identifying various medical instruments based on the specific orientation of tracking markers 260 relative to one another on a marker assembly 445. Other identifiers (e.g. a QR code or barcode) may also provide information such as the tools central point, the tools central axis, etc.

In some embodiments the tracking markers 206 are reflectosphere markers. Typically, a minimum of three spherical tracking markers 206 are attached on medical instrument 360 to enable tracking in three dimensions. In other embodiments, as shown in FIG. 4C and FIG. 4D, four spherical tracking markers 206 are attached to each medical instrument 360.

An advantageous feature of an optical tracking device is the selection of markers that can be segmented easily and therefore easily detected by tracking camera 307. For example, infrared (IR)-reflecting markers and an IR light source can be used. Such an apparatus is known, for example, from tracking devices such as the “Polaris™” system available from Northern Digital™.

However, optical tracking devices, such as tracking camera 307 require tracked instruments to remain in the field of view of the camera, such that at least three tracking markers 206 are in the line-of-sight of the camera. During a medical procedure, user 201 may move the tracked instruments partially or completely outside the field of view of the camera, or may partially or completely obscure one or more tracking markers. The user's attention and view during the medical procedure may be focused on any number of things in the OR, including access port 12, clinical tool 470, display 311, or any part of patient 200. Accordingly, user 201 may be unaware that the tracked instruments are partially or completely outside the field of view of the camera, or that the user is partially or completely obscuring one or more tracking markers. Accordingly, indicator device 400 may be attached to any one of the tracked medical instruments 360 or untracked medical instruments (as shown in FIG. 4C and 4D). Indicator device 400 may have a visual indicator positioned in view of user 201 during the medical procedure. Indicator device 400 may be configured to receive, from medical navigation system 205, data indicating a quality of the tracking of the tracked medical instruments 360. Indicator device 400 may be configured to display, using visual indicator 408, data indicating the quality of the tracking. The data displayed on the visual indicator 408 may thus aid user 201 in recognizing that one or more tracked instruments are partially or completely outside the field of view of the camera, or that the user is partially or completely obscuring one or more tracking markers. User 201 may thus move the tracked instruments to an appropriate position for tracking without shifting his or her view away from the tracked instrument, thereby reducing distributions to the flow of the medical procedure.

Each medical instrument 360 may have an indicator device 400 attached thereto. In some embodiments, only tracked medical instruments have an indicator device 400 attached thereto. Each indicator device 400 may therefore display the quality of the tracking of only the instrument 360 attached to the indicator device. In other embodiments, each indicator device 400 may display the quality of the tracking of more than one instrument 360. Indicator device 400 may display the quality of the tracking of each instrument 360 independently (e.g. quality of tracking of access port 12 and quality of tracking of clinical instrument 470 displayed independently of one another. In other embodiments, indicator device 400 may display the quality of the tracking of all instruments as a single indicia.

In one embodiment, the tracked medical instrument 360 is a clinical tool, such as access port 12 or clinical tool 470. As shown in FIG. 4C, access port 12 is configured to provide the user with access to internal tissue during the medical procedure via distal end 407 and has a proximal end 409 in view of user 201. Accordingly, in one embodiment, indicator device 400 is attached to proximal end 409 of access port 12. Similarly, as shown in FIG. 4D, clinical tool 470 has a distal end 407 configured to interface with the internal tissue (e.g. via port 12) during the medical procedure, and a handle 411 at a proximal end 409 thereof. Accordingly, in one embodiment, indicator device 400 is attached to proximal end 409 of clinical tool 470.

In another embodiment, as shown in FIG. 4E, indicator device 400 is attached to tracking camera 307. Since tracking camera 307 is directed towards user 201 and patient 202, indicator device 400 may be easily be positioned in view of a user during the medical procedure. Indicator device 400 may be placed on the front plate of tracking camera 307, or integrated into the body/encasing of tracking camera 307.

During use, medical navigation system 200 may send, via wireless transceiver 314, to indicator device 400, via wireless transceiver 410, data indicating the quality of the tracking of one or multiple tracked instruments 360. Indicator device 400 may operate under programmed computer-code stored in memory 404 to display the data received indicating the quality using visual indicator 408. In some embodiments, the data indicating the quality is sent from medical navigation system 200 in real-time and continuously. (Data may be sent in near real-time and near continuously, as may be understood by those of ordinary skill in the art. The data may be sent as a stream of data at regular or semi-regular intervals, thereby providing near real-time updates of quality of tracking).

In one embodiment, the quality of the tracking is indicative of a level of connectivity between one or more tracking instruments 360 and tracking camera 307. In one embodiment, visual indicator 408 of indicator device 400 displays the overall quality of tracking of the tracked instrument 360 to which indicator device 400 is attached. For example, if all markers 206 attached to the tracked instrument 360 are within the field of view and light-of-sight of tracking camera 307, then a ‘high’ quality indicator may be displayed in association with that instrument. Similarly, if at least three markers 206 attached to the tracked instrument 360 are within the field of view and light-of-sight of tracking camera 307, then a ‘medium’ quality indicator may be displayed in association with that instrument. Finally, if less than three markers 206 attached to the tracked instrument 360 are within the field of view and light-of-sight of tracking camera 307, then a low' quality indicator may be displayed in association with that instrument.

Since each instrument has multiple optical tracking markers 206, data for each marker 206 attached to a tracked instrument 360 may be provided by medical navigation system 205 separately. Visual indicator 408 may display the quality of the tracking of each one of the markers individually. Accordingly, an indicator may be displayed for each marker 206 attached to instrument 360. The added level of granularity provides user 201 with immediate information regarding each marker individually; thereby allowing user 201 to bring any particular marker into the optimal position for tracking by tracking camera 307 quickly. For example, if a marker is partially obscured from the light-of-sight of tracking camera 307, then a ‘medium’ quality indicator may be displayed in association with that marker. Similarly, if the marker is fully within the light-of-sight of tracking camera 307, then a ‘high’ quality indicator may be displayed in association with that marker. Finally, if the marker is fully obscured from the light-of-sight of tracking camera 307, then a low' quality indicator may be displayed in association with that marker.

In one embodiment colors are used to indicate quality in the visual indicator 408. For example, visual indicator 408 may display a green colored indicator if the level of connectivity is ‘high’. Similarly, visual indicator 408 may display a yellow colored indicator if the level of connectivity is ‘medium’. Finally, visual indicator 408 may display a red colored indicator if the level of connectivity is ‘low’. In one embodiment, a number of bars may be used to indicate quality in the visual indicator 408. The bars may be in black & white, greyscale, or may be colored using the red, yellow, and green color scheme described (or other color scheme).

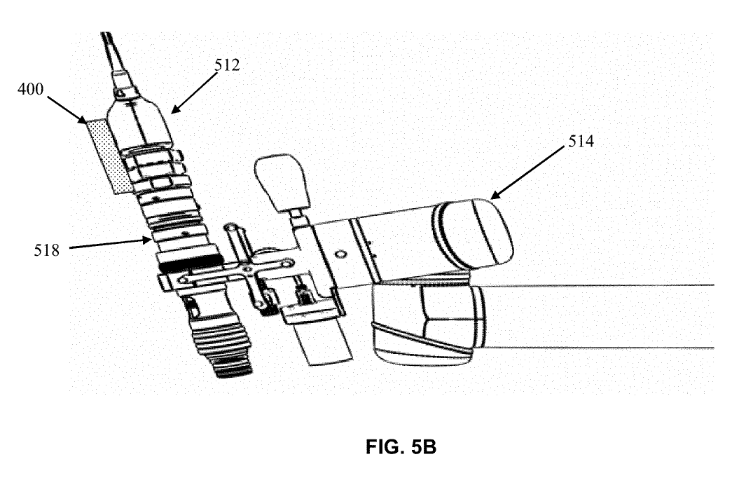

FIG. 5A shows an exemplary navigation system environment 210 in which indicator device 400 may operate. As shown, navigation system environment 210 includes the components of navigation system environment 200 of FIG. 4A and an intelligent positioning system 508. Intelligent positioning system 508 comprises an automated arm 514, a lifting column 516, and an end effector 518. End effector 518 is placed in proximity to patient 202. Lifting column 516 is connected to a frame of intelligent positioning system 508. The proximal end of automated mechanical arm 514 (further known as automated arm herein) is connected to lifting column 516. In other embodiments, automated arm 514 may be connected to a horizontal beam, which is then either connected to lifting column 516 or directly to frame of the intelligent positioning system 508. Automated arm 514 may have multiple joints to enable 5, 6 or 7 degrees of freedom.

End effector 518 is attached to the distal end of automated arm 514. End effector 518 may accommodate a plurality of instruments or tools that may assist surgeon 201 in his procedure. End effector 518 is shown as holding an external scope, however it should be noted that this is merely an example and alternate devices may be used with the end effector 518 such as a wide field camera, microscope and OCT (Optical Coherence Tomography) or other imaging instruments. Further, indicator device 400 may be attached to end effector 518, as shown in FIG. 5B. Since tracking camera 307 is in close proximity to patient 202 and user 201, indicator device 400 may be easily be positioned in view of a user during the medical procedure.

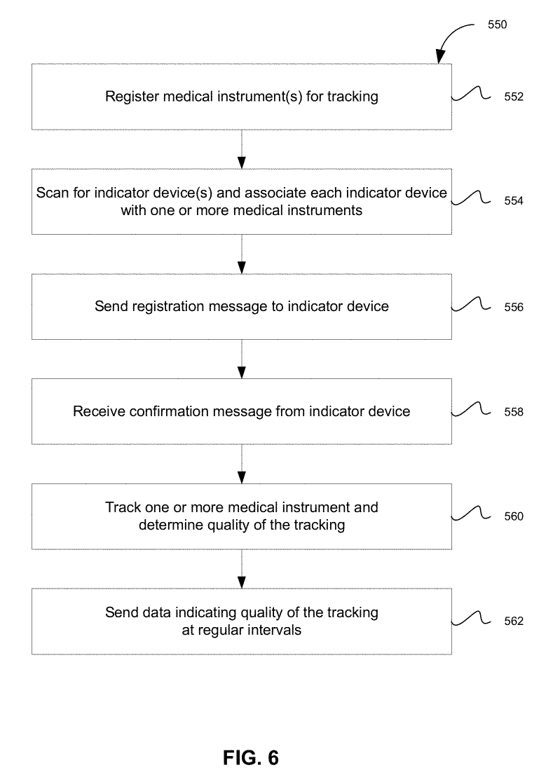

Reference is now made to FIG. 6 which illustrates a flowchart of an example method 550 for providing indicator data during a medical procedure to indicator device 400. Method 550 may be implemented by fully or partially by medical navigation system 205. Method 550 may be carried out by software executed, for example, by processor(s) 302, or alternatively, by various hardware components. Method 550 may contain additional or fewer processes than shown and/or described, and may be performed in a different order. Computer-readable code executable by the processor(s) 302 to perform method 550 may be stored in a computer-readable medium such as memory 304.

At block 552, medical navigation system 205 registers one or more medical instruments 360 for tracking. Since each instrument has multiple optical tracking markers 206, medical navigation system 205 can detect the position of each instrument relative to a reference device (not shown).

Prior to tracking, medical navigation system 205 initializes the indicator devices 400. At block 554, medical navigation system 205 scans, using wireless transceiver 314, for indicator devices 400 operating in medical navigation environment 200 and within range of transceiver 314. During the initialization phase, indicator devices 400 may transmit, via wireless transceiver 410 of the indicator device, a beacon signal, which may be detected by wireless transceiver 314 of medical navigation system 205.

In one embodiment, indicator devices 400 are in ‘sleep’ listen only mode until a ‘wake up’ signal is received. Medical navigation system 205 may transmit a ‘wake up’ signal during the initialization phase to activate the transmitters of indicator devices 400. Upon activation, indicator devices 400 in the vicinity of medical navigation system 205 will transmit the beacon signal. Such an initialization process allows for indicator devices 400 to conserve power until after medical navigation system 205 is activated.

The beacon signal or signals received at medical navigation system 205 may also include a unique identifier associated with the indicator device 400. The unique identifier may be used by medical navigation system 205 to associate each indicator device with one or more medical instruments 360. For example, a first unique identifier in the beacon signal may indicate to medical navigation system 205 that the indicator device 400 is attached to access port 12, whereas a second unique identifier in the beacon signal may indicate that the indicator device 400 is attached to clinical tool 470. Medical navigation system 205 may build an in-memory association table or association database associating each indicator device 400 detected with a medical instrument 360. The association table may then be used to send to each indicator device 400 quality data associated with the medical instrument to which the indicator device 400 is attached. Alternatively, in other embodiments, medical navigation system 205 may send the quality data to all indicator devices (i.e. without targeting a single indicator device), but may include as a prefix to the quality data the unique identifier associated with the indicator device or devices to which the data is relevant.

Also during the initialization phase, medical navigation system 205 and the indicator devices may exchange handshaking messages. At block 556, medical navigation system 205 may send to each indicator device detected during the initialization phase a registration message, and at block 558, medical navigation system 205 may receive from at least some of the indicator devices detected a confirmation message. In some embodiments, the indicator devices send the registration message to medical navigation system 205, and medical navigation system 205 responds by sending a confirmation message to each indicator device.

After the initialization phase is complete, a medical procedure may begin. During the medical procedure, at block 560, medical navigation system 205 may track one or more medical instruments and determine the quality of the tracking of each medical instrument. Medical navigation system 205 may track each medical instrument by identifying the location of tracking markers 206 attached to the medical instrument, as previously discussed, using tracking camera 307.

Medical navigation system 205 may measure the quality of the tracking for each medical instrument based on any number of factors, taken in isolation or in combination. Such factors include the number of tracking markers 206 that are in the field of view and line-of-sight of tracking camera 307 and whether tracking markers 206 are partially or fully obscured, or fully visible to the tracking camera 307.

Medical navigation system 205 may determine/compute a quality of tracking value for each medical instrument and/or for each tracking marker 206 associated with each medical instrument. Medical navigation system 205 may then store the quality of tracking data in a tracking quality table in memory 304, and/or in a database or file in storage 312. Quality of tracking data may be collected and stored over a long period of use to allow for system debugging and for system performance characterization.

Medical navigation system 205 may track medical instruments in real-time or in near real-time. Accordingly, medical navigation system 205 may update the quality of the tracking table and/or database at regular intervals. In the quality table, medical navigation system 205 may list each quality data value as being associated with a particular medical instrument using the instrument's unique identification, and may further list the associated indicator device based on the unique identifier for the indicator device.

Furthermore, at block 562, medical navigation system 205 may send, using wireless transceiver 314, data indicating data indicating quality of the tracking to indicator devices 400. Medical navigation system 205 may selectively send quality data associated with a particular medical instrument to only the associated indicator device or devices, or may send the quality data to all indicator devices active in the environment 200. Upon receipt of the quality data, each indicator device 400 may display, on visual indicator 408, the quality of the tracking of the one or more instruments associated with the indication device.

Medical navigation system 205 may also send the quality data at regular or semi-regular intervals. Each indicator device 400 will update the indicia of quality of tracking being displayed on visual indicator 408 when new data is sent by medical navigation system 205 and received at the indicator device. This is required to maintain the ‘freshness’ of the data.

In other embodiments, the updated data may be sent only when the data is changed. In other words, if no change occurs in the quality data associated with tracking a particular medical instrument, then medical navigation system 205 will not send any updates. This will reduce the number of signals being send, allowing medical navigation system 205 to be more responsive to updates in quality, and further will reduce the risks of signal interference and failed transmissions associated with wireless communications.

The above-described embodiments are intended to be examples only. Those of skill in the art may affect alterations, modifications, and variations to the particular embodiments without departing from the scope of the application. The teachings of the present disclosure are intended to cover and embrace all suitable changes in technology.

The steps and/or operations in the flowcharts and drawings described herein are for purposes of example only. There may be many variations to these steps and/or operations without departing from the teachings of the present disclosure. For instance, the steps may be performed in a differing order, or steps may be added, deleted, or modified.

While the present disclosure is described, at least in part, in terms of methods, a person of ordinary skill in the art will understand that the present disclosure is also directed to the various components for performing at least some of the aspects and features of the described methods, be it by way of hardware components, software or any combination of the two, or in any other manner. Moreover, the present disclosure is also directed to a pre-recorded storage device or other similar computer readable medium including program instructions stored thereon for performing the methods described herein.

The present disclosure may be embodied in other specific forms without departing from the subject matter of the claims. The described example embodiments are to be considered in all respects as being only illustrative and not restrictive. The present disclosure intends to cover and embrace all suitable changes in technology. The scope of the present disclosure is, therefore, described by the appended claims rather than by the foregoing description. The scope of the claims should not be limited by the embodiments set forth in the examples, but should be given the broadest interpretation consistent with the description as a whole.

1. A medical navigation system for use during a medical procedure, the medical navigation system comprising:
a tracking camera configured to track one or more instruments during the medical procedure, the one or more instruments being in communication with the tracking camera; and
an indicator device attached to at least one of the one or more instruments, and having a visual indicator positioned in view of a user during the medical procedure,
the indicator device being configured to receive, from the medical navigation system, data indicating a quality of the tracking of the one or more instruments, and to display, using the visual indicator, the data indicating the quality of the tracking.

2. The medical navigation system of claim 1, wherein the indicator device includes a wireless transceiver for communicating with the medical navigation system.

3. The medical navigation system of claim 2, wherein the indicator device includes any one of a light-emitting diode and a display.

4. The medical navigation system of claim 1, wherein the visual indicator indicates the quality of the tracking each of one of the one or more instruments independently from one another.

5. The medical navigation system of claim 4, wherein for each of the one or more tracked tools the quality of the tracking is indicative of a level of connectivity between the instrument and the tracking camera.

6. The medical navigation system of claim 5, wherein each of the one or more instruments has a plurality of tracking markers attached thereto, and wherein the visual indicator indicates the quality of the tracking of each one of the plurality of tracking markers.

7. The medical navigation system of claim 1, wherein one of the one or more instruments is a clinical tool.

8. The medical navigation system of claim 7, wherein the clinical tool is an access port having a distal end configured to interface with the internal tissue during the medical procedure, and a proximal end in view of the user, and wherein the indicator device is attached to the proximal end of the access port.

9. The medical navigation system of claim 7, wherein the clinical tool has a distal end configured to interface with internal tissue during the medical procedure, and a handle at a proximal end thereof, and wherein the indicator device is attached to the proximal end of the clinical tool.

10. A method for providing indicator data during a medical procedure, the method comprising:
tracking, using a tracking camera, one or more instruments during the medical procedure, at least one of the one or more instruments having an indicator device attached thereto and having a visual indicator positioned in view of a user during the medical procedure;
determining a quality of the tracking of the one or more instruments;
sending, to the indicator device, indicator data indicating the quality of the tracking of one or more instruments; and
displaying, on the visual indicator, the quality of the tracking of the one or more instruments.

11. The method of claim 10, wherein the wherein the visual indicator indicates the quality of the tracking of each one of the one or more instruments independently from one another.

12. The method of claim 11, wherein each one of the one or more instruments has an associated indicator device, and wherein the associated indicator device displays the quality of the tracking of the instrument associated therewith.

13. The method of claim 12, wherein the quality of the tracking is indicative of a level of connectivity between a tracked instrument and the tracking camera.

14. The method of claim 13, wherein the tracked instrument has a plurality of tracking markers attached thereto.

15. The method of claim 14,
wherein the level of connectivity is high when all of the tracking markers attached to the tracked instrument are in the field of view of the tracking camera, and
wherein the level of connectivity is in the medium range when at least three of the tracking markers attached to the tracked instrument are in the field of view of the tracking camera, and
wherein the level of connectivity is low when less than three of the tracking markers attached to the tracked instrument are in the field of view of the tracking camera.

16. The method of claim 14, wherein the visual indicator displays a green colored indicator if the level of connectivity is high, a yellow colored indicator if the level of connectivity is in a medium range, and a red colored indicator if the level of connectivity is poor.
